(19) United States
(12) Patent Application Publication
(10) Pub. No.: US 2018/0171262 A1
(43) Pub. Date: Jun. 21, 2018

(54) NOVEL AROMA CHEMICALS HAVING A 1,2,2-TRIMETHYLCYCLOPENTAN-1-YL MOIETY

(71) Applicant: BASF SE, Ludwigshafen (DE)

(72) Inventors: Stefan RÜDENAUER, Weinheim (DE); Ralf PELZER, Fürstenberg (DE); Vijay Narayanan SWAMINATHAN, Chennai (IN); Shrirang HINDALEKAR, Mumbai (IN); Sadanand ARDEKAR, Kalyan East (IN)

(21) Appl. No.: 15/577,570

(22) PCT Filed: May 27, 2016
(86) PCT No.: PCT/EP2016/061962

§ 371 Date: Nov. 28, 2017

(30) Foreign Application Priority Data
May 29, 2015 (IN) 2703/CHE/2015
Jul. 20, 2015 (EP) 15177530.1
Apr. 7, 2016 (IN) 201641012295

(51) Int. Cl. C11B 9/00; C07C 47/225; C07C 33/12; C07C 49/647; C07C 31/135; C07C 45/45; C07C 29/143; A23L 27/20

(52) U.S. Cl. CPC C11B 9/003; C07C 47/225; C07C 33/12; C07C 49/647; A23V 2002/00; C07C 45/45; C07C 29/143; A23L 27/203; C07C 2601/08; C07C 31/135

(57) ABSTRACT

The present invention relates to novel compounds of general formulae (la), (lb) and (Ic) and to the stereoisomers thereof. The compounds are useful as a fragrance or as flavor as they have a sandalwood like scent. The invention also relates to a method for imparting or modifying a scent or a flavor to a composition by including said compounds into such composition, to a fragrance containing composition and/or a fragrance material containing said compound and to a process for preparing these compounds. X is C(R⁴)—OH or C═O; R¹ is selected from the group consisting of hydrogen, C₁-C₄-alkyl, C₂-C₄-alkenyl and C₃-C₄-cycloalkyl, R² is selected from the group consisting of hydrogen, C₁-C₄-alkyl, C₂-C₄-alkenyl and C₃-C₄-cycloalkyl, R³ is C₁-C₄-alkyl, R^{3a} is selected from the group consisting of hydrogen and C₁-C₄-alkyl, R^{3b} is hydrogen or together with R^{3a} is CH₂; R⁴ is selected from the group consisting of hydrogen and C₁-C₄-alkyl.

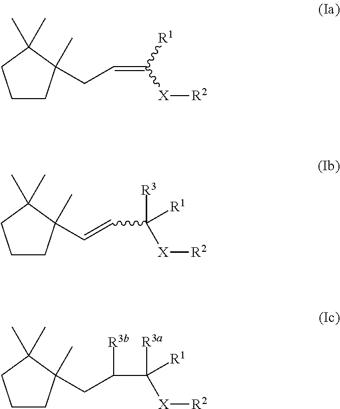

The present invention relates to novel compounds having a 1,2,2-trimethylcyclopentan-1-yl moiety and their use as a fragrance or as flavor. The invention also relates to a method for imparting or modifying a scent or a flavor to a composition by including said compounds into such composition, to a fragrance containing composition and/or a fragrance material containing said compound and to a process for preparing these compounds.

### BACKGROUND OF THE INVENTION

Aroma chemicals, i.e. fragrances and flavors, are of great interest, especially in the field of cosmetics and also laundry and cleaning detergents. Fragrances of natural origin are mostly expensive, often limited in their available amount and, on account of fluctuations in environmental conditions, are also subject to variations in their content, purity etc. It is therefore of great interest to be able to replace, at least partially, fragrances of natural origin with synthetically obtainable substances. Often, in this connection, the natural substance is not replicated chemically, but chemically synthesized compounds are selected as substitutes for natural substances on account of their odor, where substitute and natural substance do not necessarily have to have a chemical-structural similarity.

However, since even small or simple changes in chemical structure, in particular the geometry or substitution pattern, may bring about massive changes in the sensory properties such as odor perception, both in terms of odor threshold and character, and also taste, the targeted search for substances with certain sensory properties such as a certain odor is extremely difficult—see C. S. Sell, Angew. Chem. Int. Ed. 2006, 45, 6254-6261. The search for new fragrances and flavorings is therefore in most cases difficult and laborious without knowing whether a substance with the desired odor and/or taste will even actually be found.

Sandalwood oil is one of the most precious perfume ingredients, ever sought after due to its distinctive orientalic, sweet and woody note that it gives to perfume compositions. Since the natural source of the oil is very limited, synthetic alternatives are of great interest to the flavor and fragrance industry.

K. Schulze et al. Monatshefte für Chemie 120 (1989) 547-559 describe that compounds of formulae A and A′,

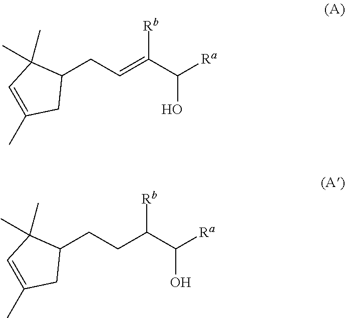

wherein R^{a} and R^{b}, independently of each other are hydrogen, methyl or ethyl, have a wood-like odor. The compounds A and A′ are prepared from campholenal (2,2,3-trimethylcyclopent-3-ene-1-acetaldehyde), which is not readily available and quite expensive.
C. Chapuis et al. Helvetica Chimica Acta 75 (1992) 1527-1546 and Helvetica Chimica Acta 89 (2006) 2638-2653 describe the preparation of compounds of the formula B

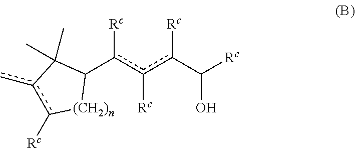

where n is 1 or 2, X is H, CH₂ or CH₃, R^{c}, independently of each other represent hydrogen, one or two lower alkyl groups and where the dashed lines indicate a single bond or a double bond. The compounds are suggested to have a sandalwood-like scent. The compounds B require expensive starting materials, which are not readily available, such as campholenal or fencholenal (2,2,4-trimethylcyclopent-3-ene-1-acetaldehyde).

### SUMMARY OF THE INVENTION

It is an object of the present invention to provide compounds, which have a pleasant odor, in particular a sandalwood-like odor, and thus are useful as novel fragrances or flavors. It should be possible to synthesize the compounds in large-scale from readily obtainable starting materials. The compounds should also be free from toxicological concerns and should in particular not contain organic halogen.

It was surprisingly found, that this objective is achieved by the compounds of the hereinafter defined formulae (Ia), (Ib) and (Ic).

Therefore, a first aspect of the present invention relates to compounds of general formulae (Ia), (Ib) and (Ic):

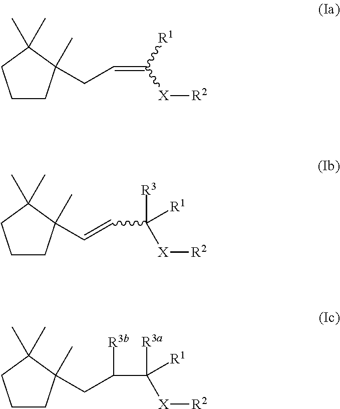

and mixtures thereof, in which
X is C(R⁴)—OH or C═O;
R¹ is selected from the group consisting of hydrogen, C₁-C₄-alkyl, C₂-C₄-alkenyl and C₃-C₄-cycloalkyl,
R² is selected from the group consisting of hydrogen, C₁-C₄-alkyl, C₂-C₄-alkenyl and C₃-C₄-cycloalkyl,
R³ is C₁-C₄-alkyl,
R^{3a} is selected from the group consisting of hydrogen and C₁-C₄-alkyl,
R^{3b} is hydrogen or together with R^{3a} is CH₂;
R⁴ is selected from the group consisting of hydrogen and C₁-C₄-alkyl,
and to the stereoisomers thereof.

The invention also relates to the use of the compounds of the general formulae (Ia), (Ib) or (Ic), or a mixture of one or more of these compounds as a fragrance or flavor.

The invention also relates to a fragrance containing composition, which contains at least one compound of the general formulae (Ia), (Ib) or (Ic), or a mixture of one or more of these compounds as a fragrance or flavor and a carrier.

The invention also relates to a method of imparting or modifying a scent or a flavor to a composition, which method comprises incorporating at least one compound of the formulae Ia, Ib or Ic or a mixture of one or more of these compounds into a composition in such an amount that imparts or modifies the scent or flavor of the composition

The invention also relates to a process for preparing a compound of the formula Ia or Ib as defined herein:
i. providing 2-(1,2,2-trimethylcyclopentyl)acetaldehyde of the formula (II)

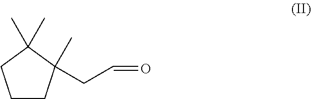

ii. reacting 2-(1,2,2-trimethylcyclopentyl)acetaldehyde with a compound of the formulae (IIIa) or (IIIb)

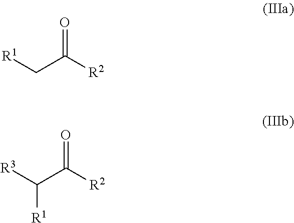

wherein R¹ R² and R³ are as defined herein, under conditions of an aldol condensation to obtain a compound of the formulae (Ia) or (Ib), wherein X is C═O,
and optionally
iii. subjecting the compound of the formulae (Ia) or (Ib), wherein X is C═O to a reduction reaction of the carbonyl group to a hydroxyl group to obtain a compound of the formulae (Ia) or (Ib), wherein X is CH—OH, or
iv. reacting the compound of the formulae (Ia) or (Ib), wherein X is C=O, with a metal organic compound R^{4a}M, wherein R^{4a} is C₁-C₄-alkyl and M is a metal atom or a metal halide radical, to obtain a compound of the formulae (Ia) or (Ib), wherein X is C(R^{4a})—OH.

The invention also relates to a process for preparing a compound of the formula (Ic) as defined herein, where R^{3b} is H, which comprises subjecting the compound of the formulae (Ia) or (Ib) to a hydrogenation of the C═C double bond.

The invention also relates to a process for preparing a compound of the formula (Ic) as defined herein, where R^{3a} and R^{3b} together form CH₂, which comprises providing a compound of the formula (Ia) and subjecting the compound of the formula (Ia) to a cyclopropanation of the C═C double bond.

### DETAILED DESCRIPTION OF THE INVENTION

In the following context, the terms alkyl, alkenyl and cycloalkyl are generic terms, which define a group of individual radicals. The prefix Cₙ-Cₘ defines the number of carbon atoms an individual radical of such groups may have.

In particular, the term C₁-C₄-alkyl defines a linear or branched saturated hydrocarbon radical, which has 1, 2, 3 or 4 carbon atoms, such as methyl ethyl, n-propyl, isopropyl, n-butyl, 2-butyl, 2-methylpropyl, and tert.-butyl (1,1-dimethylethyl).

In particular, the term C₂-C₄-alkenyl defines a linear or branched ethylenically unsaturated hydrocarbon radical, which has 2, 3 or 4 carbon atoms, such as ethenyl, 1-propenyl, propen-2-yl, buten-1-yl, buten-2-yl, buten-3-yl, 1-methylpropen-1-yl, 2-methylpropen-1-yl, 1-methylpropen-2-yl and 2-methylpropen-2-yl.

In particular, the term C₃-C₄-cycloalkyl defines a saturated, cyclic hydrocarbon radical, which has 3 or 4 carbon atoms, namely cyclopropyl, 1-methylcyclopropyl, 2-methylcyclopropyl or cyclobutyl.

In particular, the term C₁-C₄-alkoxy defines an oxygen bound linear or branched saturated radical, which has 1, 2, 3 or 4 carbon atoms, such as methoxy, ethoxy, n-propyloxy, isopropyloxy, n-butoxy, 2-butoxy, 2-methylpropyloxy, and tert.-butoxy (1,1-dimethylethoxy).

As pointed out above have an intensive and pleasant odor, in particular a sandalwood-like odor or scent, and thus are useful as novel fragrances or flavors.

Intensive odor impressions are to be understood as meaning those properties of aroma chemicals which permit a precise perception even in very low gas-space concentrations. The intensity can be ascertained via a threshold-value determination. A threshold value is the concentration of a substance in the relevant gas space at which an odor impression can just still be perceived by a representative test panel, although it no longer has to be defined. The substance class known as probably one of the most odor-intensive, i.e. those with very low threshold values, are thiols, whose threshold value is in the ppb/m³ range. It is the aim of the search for new aroma chemicals to find substances with the lowest possible threshold value in order to permit the lowest possible use concentration. The closer one comes to this target, the more one talks of “intensive” odor substances or aroma chemicals.

“Pleasant odors” or “Advantageous sensory properties” are hedonic expressions which describe the niceness and preciseness of an odor impression conveyed by an aroma chemical.

“Niceness” and “preciseness” are terms which are familiar to the person skilled in the art, a perfumer. Niceness generally refers to a spontaneously brought about, positively perceived, pleasant sensory impression. However, “nice” does not have to be synonymous with “sweet”. “Nice” can also describe the odor of musk or sandalwood. “Preciseness” generally refers to a spontaneously brought about sensory impression which—for the same test panel—brings about a reproducibly identical reminder of something specific.

For example, a substance can have an odor which is spontaneously reminiscent of that of an “apple”: the odor would then be precisely of “apple”. If this apple odor were very pleasant because the odor is reminiscent, for example, of a ripe and sweet apple, the odor would be termed “nice”. However, the odor of a typically tart apple can also be precise. If both reactions arise upon smelling the substance, in the example thus a nice and precise apple odor, then this substance has particularly advantageous sensory properties.

The compounds of the formulae (Ia) and (Ib) have a C═C carbon double bond. The double bond in (Ia) may have E- or Z-configuration with respect to the 1,2,2-trimethylcyclopenan-1-ylmethyl radical and the moiety X—R². The invention relates to both the Z-stereoisomer of (Ia) and the E-stereoisomer of (Ia) and to mixtures of the E- and Z-stereoisomer of (Ia). Likewise, the double bond in (Ib) may have E- or Z-configuration with respect to the 1,2,2-trimethylcyclopenan-1-yl radical and the moiety C(R¹R³)—X—R². The invention relates to both the Z-stereoisomer of (Ib) and the E-stereoisomer of (Ib) and to mixtures of the E- and Z-stereoisomer of (Ib).

The compounds of the formulae (Ia), (Ib) and (Ic) have one or more centers of chirality. The present invention relates to pure stereoisomers, diastereomers and mixtures of stereoisomers or diastereoisomers. One center of chirality in formulae (Ia), (Ib) and (Ic) is the carbon atom in the 1-position of the 2,2-dimethyl-1-methylcyclopentyl moiety, which may have S or R configuration. If this carbon atom is the only center of chirality, the invention relates to racemic mixtures as well as to non-racemic mixtures and to the pure enantiomers.

In the compounds of formulae (Ia), (Ib) and (Ic), wherein X is C(R⁴)—OH, a further center of chirality may be the carbon atom, which carries the OH group, provided that R² is different from R⁴. In case R² is different from R⁴, the carbon atom carrying OH may have S or R configuration. In this case the compounds of formulae (Ia), (Ib) and (Ic) have at least two centers of chirality and these compounds may be present as 4 diastereomers, namely the RS-, SR-, RR- and SS-diastereomers. The present invention relates the pure diastereomers as well as to mixtures of these diastereomers.

In the compounds of formulae (Ib) and (Ic), a further center of chirality may be the carbon atom, which carries R¹. In case R¹ is different from R^{3a} or R^{3a}, respectively, the carbon atom carrying R¹ may have S or R configuration. In this case the compounds of formulae (Ib) and (Ic) have at least two centers of chirality and these compounds may be present as 4 diastereomers, namely the RS-, SR-, RR- and SS-diastereomers. The present invention relates the pure diastereomers as well as to mixtures of these diastereomers.

In terms of the present invention, the term “pure enantiomer” has to be understood as a non-racemic mixture of a specific compound, where the desired enantiomer is present in an enantiomeric excess of >90% ee.

In terms of the present invention, the term “pure diastereomer” has to be understood as a mixture of the diastereomers of a specific compound, where the desired diastereomer is present in an amount of >90% based on the total amount of diastereomers of said compound.

As regards the carbon atom in the 1-position of the 2,2-dimethyl-1-methylcyclopentyl moiety of the formulae (Ia), (Ib) and (Ic), this carbon atom may have (R) or (S) configuration. A particular embodiment of the present invention relates the compounds of the formulae (Ia), (Ib) and (Ic), where carbon atom in the 1-position of the 2,2-dimethyl-1-methylcyclopentyl moiety of the formulae (Ia), (Ib) and (Ic) has predominately (S) configuration, in particular where the ratio (S)/(R) is at least 4:1, in particular at least 9:1, especially at least 20:1. Another particular embodiment of the present invention relates the compounds of the formulae (Ia), (Ib) and (Ic), where carbon atom in the 1-position of the 2,2-dimethyl-1-methylcyclopentyl moiety of the formulae (Ia), (Ib) and (Ic) has predominately (R) configuration, in particular where the ratio (R)/(S) is at least 4:1, in particular at least 9:1, especially at least 20:1.

A further particular embodiment of the present invention relates the compounds of the formulae (Ia), (Ib) and (Ic), where carbon atom in the 1-position of the 2,2-dimethyl-1-methylcyclopentyl moiety of the formulae (Ia), (Ib) and (Ic), where the ratio (S)/(R) is in the range from 1:4 to 4:1, in particular in the range from 1:2 to 2:1 and, especially in the range from 1:1.5 to 1.5:1.

A particular group IA of embodiments of the invention relates to compounds of the formula (Ia), including their stereoisomers and mixtures of the compounds of formula (Ia), in particular mixtures of stereoisomers of the formula (Ia). Preference is given to compounds of the formula (Ia), wherein the total number of carbon atoms in R¹ and R² is at most 6, in particular at most 5 or at most 4. Preference is also given to compounds of the formula (Ia), wherein X is C(R⁴)—OH and wherein the total number of carbon atoms in R¹, R² and R⁴ is at most 8, in particular at most 6 and especially at most 5 or at most 4. Particular preference is given to compounds of the formula (Ia), wherein the variables R¹ and R² and R⁴, if present, individually or in particular in combination have preferably one of the following meanings:
R¹ is in particular selected from hydrogen and C₁-C₄-alkyl and especially from hydrogen, methyl or ethyl;
R² is in particular selected from hydrogen and C₁-C₄-alkyl and especially from hydrogen, methyl or ethyl;
R⁴ if present, is in particular hydrogen, methyl or ethyl, especially hydrogen.

A particular group IA-1 of embodiments relates to compounds of formula (Ia), wherein X is C═O and wherein R¹ and R² are as defined herein and have in particular one of the preferred meanings.

Another particular group IA-2 of embodiments relates to compounds of formula (Ia), wherein X is CH—OH and wherein R¹ and R² are as defined herein and have in particular one of the preferred meanings.

A further particular group IA-3 of embodiments relates to compounds of formula (Ia), wherein X is C(R^{4a})—OH and wherein R¹ and R² are as defined herein and have in particular one of the preferred meanings and wherein R^{4a} is C₁-C₄-alkyl, in particular methyl or ethyl.

A particular group IA′ of embodiments of the invention relates to mixtures of compounds of the formula (Ia), including their stereoisomers, with a mixture of a compound of formula (Ic), in particular mixtures of compounds of the formulae (Ia) and (Ic), wherein R¹ in formula (Ia) is identical with R¹ in formula (Ic), R² in formula (Ia) is identical with R² in formula (Ic), and R^{3a} is hydrogen.

Examples of the compounds of formula (Ia) include, but are not limited to:
4-(1,2,2-trimethylcyclopentyl)but-2-enal,
2-methyl-4-(1,2,2-trimethylcyclopentyl)but-2-enal,
2-ethyl-4-(1,2,2-trimethylcyclopentyl)but-2-enal,
5-(1,2,2-trimethylcyclopentyl)pent-3-en-2-one,
3-methyl-5-(1,2,2-trimethylcyclopentyl)pent-3-en-2-one,
3-ethyl-5-(1,2,2-trimethylcyclopentyl)pent-3-en-2-one,
6-(1,2,2-trimethylcyclopentyl)hex-4-en-3-one,
4-methyl-6-(1,2,2-trimethylcyclopentyl)hex-4-en-3-one,
4-(1,2,2-trimethylcyclopentyl)but-2-en-1-ol,
2-methyl-4-(1,2,2-trimethylcyclopentyl)but-2-en-1-ol,
2-ethyl-4-(1,2,2-trimethylcyclopentyl)but-2-en-1-ol,
5-(1,2,2-trimethylcyclopentyl)pent-3-en-2-ol,
3-methyl-5-(1,2,2-trimethylcyclopentyl)pent-3-en-2-ol,
3-ethyl-5-(1,2,2-trimethylcyclopentyl)pent-3-en-2-ol, and
6-(1,2,2-trimethylcyclopentyl)hex-4-en-3-ol,
including their stereoisomers and mixtures thereof.

Another particular group IB of embodiments of the invention relates to compounds of the formula (Ib), including their stereoisomers and mixtures of the compounds of formula (Ib), in particular mixtures of stereoisomers of the formula (Ib). Preference is given to compounds of the formula (Ib), wherein the total number of carbon atoms in R¹, R², R³ and R⁴ is at most 8, in particular at most 6, especially at most 5 or at most 4. Particular preference is given to compounds of the formula (Ib), wherein the variables R¹, R², R³ and R⁴ individually or in particular in combination have preferably one of the following meanings:
R¹ is in particular selected from hydrogen and C₁-C₄-alkyl and especially from hydrogen, methyl or ethyl;
R² is in particular selected from hydrogen and C₁-C₄-alkyl and especially from methyl or ethyl;
R³ is in particular selected from C₁-C₄-alkyl and especially from methyl or ethyl.
R⁴ if present, is in particular hydrogen, methyl or ethyl, especially hydrogen.

A particular group of embodiments of the compounds of formula (Ib) relates to those compounds, wherein X is C═O and wherein R¹, R² and R³ are as defined herein and have in particular one of the preferred meanings.

Another particular group of embodiments of the compounds of formula (Ib) relates to those compounds, wherein X is CH—OH and wherein R¹, R² and R³ are as defined herein and have in particular one of the preferred meanings.

Another particular group of embodiments of the compounds of formula (Ib) relate to those compounds, wherein X is C(R^{4a})—OH and wherein R¹, R² and R³ are as defined herein and have in particular one of the preferred meanings and where R^{4a} is C₁-C₄-alkyl, in particular methyl or ethyl.

Examples of the compounds of formula (Ib) include, but are not limited to:
2,2-dimethyl-4-(1,2,2-trimethylcyclopentyl)but-3-enal,
2,2-dimethyl-4-(1,2,2-trimethylcyclopentyl)but-3-en-1-ol,
3,3-dimethyl-5-(1,2,2-trimethylcyclopentyl)pent-4-en-2-one, and
3,3-dimethyl-5-(1,2,2-trimethylcyclopentyl)pent-4-en-2-ol,
including their stereoisomers and mixtures thereof.

A further particular group IC of embodiments of the invention relates to compounds of the formula (Ic), where R^{3b} is hydrogen, including their stereoisomers and mixtures of the compounds of formula (Ic), in particular mixtures of stereoisomers of the formula (Ic). Preference is given to compounds of the formula (Ic), where R^{3b} is hydrogen, wherein the total number of carbon atoms in R¹, R² and R^{3a} is at most 6, in particular at most 5 or at most 4. Particular preference is given to compounds of the formula (Ic), where R^{3b} is hydrogen and wherein the variables R¹, R² and R^{3a} and R⁴, if present, individually or in particular in combination have preferably one of the following meanings:
R¹ is in particular selected from hydrogen and C₁-C₄-alkyl and especially from hydrogen, methyl or ethyl;
R² is in particular selected from hydrogen and C₁-C₄-alkyl and especially from hydrogen, methyl or ethyl;
R^{3a} is in particular selected from hydrogen and C₁-C₄-alkyl and especially from hydrogen, methyl or ethyl;
R⁴ if present, is in particular hydrogen, methyl or ethyl, especially hydrogen.

A particular group of embodiments of the compounds of formula (Ic), where R^{3b} is hydrogen, relates to those compounds, wherein X is C═O and wherein R¹, R² and R³ are as defined herein and have in particular one of the preferred meanings.

Another particular group of embodiments of the compounds of formula (Ic), where R^{3b} is hydrogen, relates to those compounds, wherein X is CH—OH and wherein R¹, R² and R³ are as defined herein and have in particular one of the preferred meanings.

Another particular group of embodiments of the compounds of formula (Ic), where R^{3b} is hydrogen, relates to those compounds, wherein X is C(R^{4a})—OH and wherein R¹, R² and R³ are as defined herein and have in particular one of the preferred meanings and where R^{4a} is C₁-C₄-alkyl, in particular methyl or ethyl.

Examples of the compounds of formula (Ic), where R^{3b} is hydrogen, include, but are not limited to:
4-(1,2,2-trimethylcyclopentyl)butanal,
2-methyl-4-(1,2,2-trimethylcyclopentyl)butanal,
2-ethyl-4-(1,2,2-trimethylcyclopentyl)butanal,
5-(1,2,2-trimethylcyclopentyl)pentan-2-one,
3-methyl-5-(1,2,2-trimethylcyclopentyl)pentan-2-one,
3-ethyl-5-(1,2,2-trimethylcyclopentyl)pentan-2-one,
6-(1,2,2-trimethylcyclopentyl)hexan-3-one,
4-methyl-6-(1,2,2-trimethylcyclopentyl)hexan-3-one,
4-(1,2,2-trimethylcyclopentyl)butan-1-ol,
2-methyl-4-(1,2,2-trimethylcyclopentyl)butan-1-ol,
2-ethyl-4-(1,2,2-trimethylcyclopentyl)butan-1-ol,
5-(1,2,2-trimethylcyclopentyl)pentan-2-ol,
3-methyl-5-(1,2,2-trimethylcyclopentyl)pentan-2-ol,
3-ethyl-5-(1,2,2-trimethylcyclopentyl)pentan-2-ol,
6-(1,2,2-trimethylcyclopentyl)hexan-3-ol,
4-methyl-6-(1,2,2-trimethylcyclopentyl)hexan-3-ol,
2,2-dimethyl-4-(1,2,2-trimethylcyclopentyl)butanal,
2,2-dimethyl-4-(1,2,2-trimethylcyclopentyl)butan-1-ol,
3,3-dimethyl-5-(1,2,2-trimethylcyclopentyl)pentan-2-one, and
3,3-dimethyl-5-(1,2,2-trimethylcyclopentyl)pentan-2-ol,
including their stereoisomers and mixtures thereof.

A further particular group IC′ of embodiments of the invention relates to compounds of the formula (Ic), where R^{3b} together with R^{3a} forms a CH₂ moiety, including their stereoisomers and mixtures of the compounds of formula (Ic), in particular mixtures of stereoisomers of the formula (Ic). In other words, the group IC′ of embodiments relates to compounds of the formula (Ic), where R^{3b} together with R^{3a} and together with the carbon atoms to which R^{3b} and R^{3a} are bound, form a cis- or trans-cyclopropane-1,2-diyl moiety. stereoisomer and mixtures of the compounds of formula (Ic), in particular mixtures of stereoisomers of the formula (Ic).

Preference is given to compounds of the embodiment IC′, i.e. to compounds of formula (Ic), where R^{3b} together with R^{3a} forms a CH₂ moiety, wherein the total number of carbon atoms in R¹ and R² is at most 6, in particular at most 5 or at most 4. Particular preference is given to compounds of the formula (Ic), where R^{3b} together with R^{3a} forms a CH₂ moiety, and wherein the variables R¹, R² and R⁴, if present, individually or in particular in combination have preferably one of the following meanings:
R¹ is in particular selected from hydrogen and C₁-C₄-alkyl and especially from hydrogen, methyl or ethyl;
R² is in particular selected from hydrogen and C₁-C₄-alkyl and especially from hydrogen, methyl or ethyl;
R⁴ if present, is in particular hydrogen, methyl or ethyl, especially hydrogen.

A particular group of embodiments of the compounds of formula (Ic), where R^{3b} together with R^{3a} forms a CH₂ moiety, relates to those compounds, wherein X is C═O and wherein R¹, R² and R³ are as defined herein and have in particular one of the preferred meanings.

Another particular group of embodiments of the compounds of formula (Ic), where R^{3b} together with R^{3a} forms a CH₂ moiety, relates to those compounds, wherein X is C(R⁴)—OH and especially where X is CH—OH, and wherein R¹, R² and R³ are as defined herein and have in particular one of the preferred meanings.

Another particular group of embodiments of the compounds of formula (Ic), where R^{3b} together with R^{3a} forms a CH₂ moiety, relates to those compounds, wherein X is C(R^{4a})—OH and wherein R¹, R² and R³ are as defined herein and have in particular one of the preferred meanings and where R^{4a} is C₁-C₄-alkyl, in particular methyl or ethyl.

Examples of the compounds of formula (Ic), where R^{3b} together with R^{3a} forms a CH₂ moiety, include, but are not limited to:
[trans-1-methyl-2-[(1,2,2-trimethylcyclopentyl)methyl]cyclopropylmethanol;
[trans-1-ethyl-2-[(1,2,2-trimethylcyclopentyl)methyl]cyclopropylmethanol;
[cis-1-methyl-2-[(1,2,2-trimethylcyclopentyl)methyl]cyclopropylmethanol; and
[cis-1-ethyl-2-[(1,2,2-trimethylcyclopentyl)methyl]cyclopropylmethanol,
including their stereoisomers and mixtures thereof.

A further particular group IAc of embodiments of the invention relates to mixtures of compounds of the formula (Ia), including their stereoisomer, with a mixture of a compound of formula (Ic), including their stereoisomers, in particular mixtures of compounds of the formulae (Ia) and (Ic), wherein R¹ in formula (Ia) is identical with R¹ in formula (Ic), R² in formula (Ia) is identical with R² in formula (Ic), and R^{3a} is hydrogen and R^{3b} is hydrogen.

A further particular group IBc of embodiments of the invention relates to mixtures of compounds of the formula (Ib), including their stereoisomer, with a mixture of a compound of formula (Ic), including their stereoisomers, in particular mixtures of compounds of the formulae (Ib) and (Ic), wherein R¹ in formula (Ib) is identical with R¹ in formula (Ic), R² in formula (Ib) is identical with R² in formula (Ic), and R³ in formula (Ib) is identical with R^{3a} in formula (Ic).

A further particular group IAd of embodiments of the invention relates to mixtures of compounds of the formula (Ia), including their stereoisomer, with a mixture of a compound of formula (Ic), including their stereoisomers, in particular mixtures of compounds of the formulae (Ia) and (Ic), wherein R¹ in formula (Ia) is identical with R¹ in formula (Ic), R² in formula (Ia) is identical with R² in formula (Ic), and R^{3a} together with R^{3b} forms CH₂.

The invention further relates to the use of the compounds of the formulae (Ia), (Ib) and (Ic), and to the use of a mixture thereof as defined above, in compositions, which typically comprise at least one aroma compound, i.e. at least one fragrance and/or flavoring. Such compositions include, for example, laundry detergents, fabric detergents, cosmetic preparations, other fragranced hygiene articles, such as diapers, sanitary towels, armpit pads, paper towels, wet wipes, toilet paper, pocket tissues, and the like, foods, food supplements, examples being chewing gums or vitamin products, fragrance dispensers, examples being room air fresheners, perfumes, pharmaceutical preparations, and also crop protection products.

Typically, these compositions are formulated by incorporating at least one compound of the formulae (Ia), (Ib) and (Ic) or one of the above defined mixtures thereof, optionally together with one or more other aroma compounds, into an existing preparation, which before comprises no aroma compound or which before comprises one or more other aroma compound different from compounds of the formulae (Ia), (Ib) and (Ic). Such compositions generally further comprise a carrier, which may be a compound, a compound mixture or other additives, which have no or no noticeable sensory properties. The carrier may as well be a compound or an additive having noticeable sensory properties, or a compound mixture comprising one or more other aroma compounds different from a compound of the formulae (Ia), (Ib) and (Ic) and optionally one or more compounds having no or no noticeable sensory properties.

In the compositions according to the present invention the at least one compound of the formulae (Ia), (Ib) and (Ic) or one of the above defined mixtures thereof are usually applied in amounts customary for formulation auxiliaries. More specifically the amount of the at least one compound of the formulae (Ia), (Ib) and (Ic) or of the above defined mixtures thereof is in the range of 0.001 to 50% by weight, in particular in the range of 0.01 to 20% by weight, especially in the range of 0.1 to 10% by weight, based on the total of the composition.

The at least one compound of the formulae (Ia), (Ib) and (Ic) and the above defined mixtures thereof preferably find use in laundry detergents and fabric detergents, in cosmetic preparations and in other fragranced hygiene articles. Particular preference is given to the use of the at least one compound of the formulae (Ia), (Ib) and (Ic) and of the above defined mixtures thereof in cosmetic preparations such as perfumes.

The invention further relates to a method of imparting or modifying a scent or a flavor to a composition, which method comprises including or incorporating at least one compound of the formulae (Ia), (Ib) and (Ic) or one of the above defined mixtures thereof into a composition in such an amount that imparts or modifies the scent or flavor of the composition. The total amount of the at least one compound of the formulae (Ia), (Ib) and (Ic) or of one of the above defined mixtures thereof required for modification depends on the nature and on the application purpose of the composition and can, therefore, vary in a wide range. Typically, the total amount of the at least one compound of the formulae (Ia), (Ib) and (Ic) or of the above defined mixtures thereof included/incorporated into the composition is in the range from 0.001 to 50% by weight, in particular in the range from 0.01 to 20%.

The intensively and precisely smelling compounds of the formulae (Ia), (Ib) and (Ic), and the mixture thereof as defined above are preferably used as fragrances. Suitable fields of application are all applications in which a certain scent is desired, whether it is to mask more unpleasant odors or to generate a certain odor or scent or certain odor notes or scent notes in a targeted manner.

Therefore, the invention further relates to a fragrance containing composition and/or a fragrance material, which contains at least one compound of the formulae (Ia), (Ib) and (Ic) or one of the above defined mixtures thereof and a carrier material.

The total concentration of the at least one compound of the formulae (Ia), (Ib) and (Ic) or of the above defined mixtures thereof in the fragrance containing composition and/or the fragrance material according to the present invention is not particularly limited. It can be changed in a wide range, depending on the purpose of their use. Generally, amounts that are customary for fragrances are used. The total amount of the at least one compound of the formulae (Ia), (Ib) and (Ic) or of the above defined mixtures thereof in the fragrance containing composition and/or the fragrance material is typically in the range from 0.001 to 20% by weight, in particular in the range from 0.01 to 10% by weight.

The carrier material may be a compound, a compound mixture or other additives having the properties as defined above. Suitable carrier materials may comprise liquid or oil-based carrier materials as well as wax-like or solid carrier materials.

Suitable liquid or oil-based carrier materials are for example selected from alcohols, such as ethanol, water, aliphatic diols and polyols having melting temperatures below 20° C., such as ethylene glycol, glycerol, diglycerol, propylene glycol, dipropylene glycol, cyclic siloxanes (silicon fluids), such as hexamethylcyclotrisiloxane or decamethylcyclopentasiloxane, plant-oils, such as fractionated coconut-oil, or esters of fatty alcohols having melting temperatures below 20° C., such as myristyl acetate or myristyl lactate, and alkyl esters of fatty acids having melting temperatures below 20° C., such as isopropyl-myristate.

Suitable wax-like or solid carrier materials are for example selected from fatty alcohols having melting temperatures above 20° C., such as myristyl alcohol, stearyl alcohol or cetyl alcohol, polyols and esters of fatty alcohol having melting temperatures above 20° C., synthetic petroleum derived waxes, such as paraffin waxes, water insoluble porous minerals, such as silica, silicates, for example talc, microporous aluminasilicate minerals (zeolites), clay minerals, for example bentonite, or phosphates for example sodium tripolyphosphate, paper, cardboard, wood, nonwoven of rayon staple fibers or fiber-fleeces.

Suitable carrier materials are for example also selected from water-soluble polymers, such as polyacrylic acid esters or quaternized polyvinyl pyrrolidone or water-alcohol-soluble polymers, such as specific thermoplastic polyesters and polyamides. The polymeric carrier material can be present in different forms, for example in form of a gel, a paste, or water insoluble solid particles, such as microcapsules or friable coatings.

Depending on the purpose of use, the carrier materials may further comprise other additives or auxiliaries, for example surfactants or mixtures of surfactants, viscosifiers, such as polyethylene glycols with a molecular weight of 400 to 20′000 Da, lubricates, binding or agglomerating agents, such as sodium silicate, dispersing agents, detergent builder salts, filler salts, pigments, dyes, optical brighteners, anti-redeposition agents and the like.

Typical applications of the composition and/or the fragrance material according to the present invention are in the field of laundry and cleaning detergents, preparations of fragrances for the human or animal body, for rooms such as kitchens, wet rooms, automobiles or heavy goods vehicles, for real or artificial plants, for clothing, for shoes and shoe insoles, for items of furniture, for carpets, for air humidifiers and air fresheners, for cosmetics such as perfumes.

The invention also includes odorant combinations which comprise at least one compound of the formulae (Ia), (Ib) and (Ic) or one of the above defined mixtures thereof as component A and at least one further compound known as an odorant or aroma substance, as component B, such as, for example, one or more of the following compounds B1 to B11:
B1: methyl dihydrojasmonate (e.g. hedione),
B2: 4,6,6,7,8,8-hexamethyl-1,3,4,6,7,8-hexahydrocyclopenta[g]benzopyran (e.g. Galaxolide™)
B3: 2-methyl-3-(4-tert-butylphenyl)propanal (Lysmeral™)
B4: 2-methyl-3-(4-isopropylphenyl)propanal (cyclamenaldehyde),
B5: 2,6-dimethyl-7-octen-2-ol (dihydromyrcenol),
B6: 3,7-dimethyl-1,6-octadien-3-ol (linalool),
B7: 3,7-dimethyl-trans-2,6-octadien-1-ol (geraniol),
B8: 2,3,8,8-tetramethyl-1,2,3,4,5,6,7,8-octahydro-2-naphthalenyl methyl ketone (Iso E Super™),
B9: alpha-hexylcinnamaldehyde,
B10: 3,7-dimethyl-6-octen-1-ol (citronellol),
B11: alpha- or beta- or delta-damascone.

Suitable formulations of odor substances are, for example, the formulations disclosed in JP 11-071312 A, paragraphs [0090] to [0092]. The formulations from JP 11-035969 A, paragraphs [0039] to [0043] are also likewise suitable.

As pointed out above, the compounds of the present invention can be prepared starting from 2-(1,2,2-trimethylcyclopentyl)acetaldehyde of the formula (II):

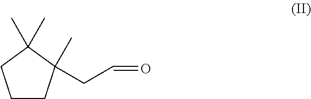

The compound of the formula (II) is readily available by reductive cyclization of citral ((2E)-3,7-dimethylocta-2,6-dienal), e.g. by reacting citral with a reducing agent in the presence of an iron-containing catalyst.

Suitable reducing agents include e.g. aryl silanes such as phenyl silane (C₆H₅—SiH₃), mono-, di- or tri-C₁-C₄-alkylsilane, such as triemethylsilane, mono-, di- or tri-C₁-C₄-alkoxysilanes, like, e.g., trimethoxysilane, triethoxysilane, and the like, also carrierbound silanes, or else alkali metal hydrides, like, e.g., sodium borohydride, lithium borohydride, or complex hydrides of aluminium, such as the alkalimetal salts of dihydrido-di-C₁-C₄-alkoxyaluminates or di-hydrido-di-(C₁-C₄-alkoxy-C₁-C₄-alkoxy)aluminates, e.g. sodium-dihydrido-di(2-methoxyethoxy)aluminate, which is commercially available as Red-Al.

Suitable iron containing catalysts include in particular iron iron (III) compounds such as tris(acetylacetonato)iron(III) or other suitable iron(III) catalyst-ligand-complexes.

The reductive cyclization of citral is preferably carried out in an organic solvent, such as an alcohol or a mixture of alcohols, including e.g. C₁-C₄-alkanols, such as methanol, ethanol, n-propanol, isopropanol, n-butanol, 2-butanol, isobutanol or tert.-butanol, and mono- or di-C₂-C₄-alkylene glykols such as ethylene glycol, propylene glycol, diethylene glycol etc.

The reductive cyclization of citral is in particular carried out by reacting citral with tris(acetylacetonato)iron (III), e.g. by the protocol described by J. C. Lo, Y. Yabe and P. S. Baran, J. Am. Chem. Soc. 2014, 136, 1304-1307.

The compounds of the formulae (Ia) or (Ib), wherein X is C═O can be prepared by reacting the compound of the formula (II) with an aldehyde or ketone of the formulae (IIIa) or (IIIb), respectively under conditions of an aldol condensation reaction. The reaction is hereinafter also termed step ii).

It is apparent that the reaction of the compound of the formula (II) with the compound of the formula (IIIa) under conditions of an aldol condensation will result in a compound of the formula (Ia), where X is C═O, while the reaction of the compound of the formula (II) with the compound of the formula (IIIb) and said conditions will result in the compound of formula (Ib), in particular, if R¹ is different from hydrogen. It is apparent that the variables R¹, R² and R³ in formulae (IIIa) and (IIIb), respectively correspond to R¹, R² and R³ in formulae (Ia) and (Ib), respectively.

The aldol condensation of step ii) may be carried out by standard aldol condensation protocols as summarized in Richard C. Larock (Ed.), “Comprehensive Organic Transformations”, 2nd Edition, Wiley VCH, 1999, p. 1317, C. H. Heathcock in Modern Synthetic Methods (Ed. R. Scheffold), VHCA, Basel 1992, pp 1-102, Organikum, 21^{st} edition, Wiley VCH 2001, pp. 518-526 and the literature cited therein.

The reaction of the compound of the formula (II) with the compound of formulae (IIIa) or (IIIb), may be carried out applying basic or acidic conditions. Preferably, the reaction of the compound of the formula (II) with the compound of formulae (IIIa) or (IIIb), respectively, is carried out in the presence of a base. The base will result in the formulation of the enolate of (IIIa) or (IIIb), respectively, which reacts with the aldehyde group of (II) followed by elimination of water.

The compound of formulae (IIIa) or (IIIb), respectively, may be used in stoichiometric amounts or in excess, based on the compound of formula (II). Preferably, the compound of formulae (IIIa) or (IIIb) are used in excess, based on the required stoichiometry, in order to achieve complete conversion of the compound of formula (II) and avoid its self-condensation. In particular the relative molar amount of the compound of formulae (IIIa) or (IIIb) to the compound of the formula (II) is from 1.1:1 to 20:1.

Suitable bases include but are not limited to alkalimetal hydrides, such as lithium, sodium or potassium hydride, alkalimetal alkanolates (sometimes termed alkalimetal alkoxides) such as sodium methoxide, potassium methoxide, sodium ethoxide, potassium ethoxide, sodium butoxide, potassium butoxide, sodium tert.butoxide or potassium tert.butoxide, alkalimetal hydroxides, such as lithium hydroxide, sodium hydroxide or potassium hydroxide, alkalimetal amides such as lithium diisopropylamide or tertiary amines, such as triethyl amine or N-methylpyrrolidin. Preferably, the base is selected from alkalimetal C₁-C₄-alkanolates and alkalimetal hydroxides, such as lithium hydroxide.

The base may be used in catalytic amounts, in stoichiometric amounts or in excess, with respect to the compound of the formulae (IIIa) or (IIIb), respectively. Preferably, the molar amount of base used in step ii) is less than the amount of the compound of formulae (IIIa) or (IIIb), respectively. In particular, the molar ratio of the base to the compound of formulae (IIIa) or (IIIb) used in step ii) is from 1:1.5 to 1:10.

Depending on the reactivity of the compound of formulae (IIIa) or (IIIb), respectively, the enolate of (IIIa) or (IIIb) may be formed in a preceding step followed by the reaction of the aldehyde of the formula (II) with the enolate of formulae (IIIa) or (IIIb). Frequently, the reaction of step ii) is performed by mixing compound of formula (II) with a compound of formulae (IIIa) or (IIIb), respectively, followed by the addition of a base.

The reaction of step ii) is preferably carried out in an organic solvent. Suitable solvents include but are not limited to alkanols, in particular to C₁-C₄-alkanols, such as methanol, ethanol, n-propanol, isopropanol, n-butanol or tert.-butanol, ethers having from 3 to 8 carbon atoms, such as tetrahydrofurane, dioxane, diethyl ether, diisopropyl ether, methyl tert. butylether and mixtures thereof. In a particular the reaction of step ii) is carried out in a C₁-C₄-alkanol or a mixture of C₁-C₄-alkanol in the presence of a base. In this embodiment, the base is preferably selected from alkalimetal C₁-C₄-alkanolates.

The compounds of the formulae (Ia) and (Ib), respectively, wherein X is C=O, can be converted to compounds of the formulae (Ia) and (Ib), respectively, wherein X is CH—OH, by selectively reducing the carbonyl group. Suitable methods for selective reduction of the carbonyl group to obtain an allyl alcohol are well known to a skilled person. The reduction of the carbonyl group may be achieved e.g. by reacting the compound of formulae (Ia) and (Ib), respectively, wherein X is C=O, with a boron hydride such as lithium, sodium or potassium tetrahydroborate or with an aluminum hydride such as lithium aluminum hydride. The reaction can be performed e.g. by analogy to the method described by S. Krishnamurthy et al. Org. Chem., 1977, 42(7), pp 1197-1201, J. C. Fuller et al. Tetrahedron Lett. 34, 1993, 257-260, B. Zeynidazeh et al. Bull. Korean Chem. Soc. 24 (3), 2003, 295-298. The reduction of the carbonyl group may also be achieved by reacting the compound of formulae (Ia) and (Ib), respectively, wherein X is C=O, with hydrogen in the presence of a transition metal catalyst, e.g. by analogy to the method described in EP 71787.

Alternatively, the compounds of the formulae (Ia) and (Ib), respectively, wherein X is C=O, can be converted into compounds of the formulae (Ia) and (Ib), respectively, wherein X is C(R^{4a})—OH, by reacting the carbonyl group of the respective compound of the formulae (Ia) or (Ib) with a metal organic reagent having a metal bound alkyl radical R4a, e.g. with metal organic compound R^{4a}M, wherein R^{4a} is C₁-C₄-alkyl, in particular methyl or ethyl, and M is a metal atom or a metal halide radical, e.g. a lithium atom or a magnesium halide radical, such as MgCl, MgBr or MgI. The reaction can be performed by analogy to well known processes of reacting carbonyl groups with metal organic compounds R^{4a}M, such as under the conditions of a Grignard Reaction—see e.g. K. Nützel, et al. Methoden Org Chem (Houben Weyl) 1973, Vol. 13/2a, pp. 49-527; J. C. Stowell, Chem. Rev. 1984, 84, 409-435, H. M. Walborsky, Acc. Chem. Res. 1990, 23, 286-293, J. F. Garst, Acc. Chem. Res. 1991, 24, 95-97, A. Fürstner, Angew. Chem. Int. Ed. Engl. 1993, 32, 164-189 and the references cited therein,

The compounds of the formula (Ic), where R^{3b} is hydrogen, can be prepared from compounds of the formulae (Ia) or (Ib) by subjecting the compound of the formulae (Ia) or (Ib) to a hydrogenation of the C═C double bond.

For example, the compounds of the formulae (Ia) and (Ib), respectively, wherein X is C═O, can be converted to compounds of the formulae (Ic), respectively, wherein X is C═O, by selectively hydrogenating the C═C double bound in (Ia) or (Ib), respectively. Suitable methods for selective hydrogenation of the C═C bond in unsaturated aldehydes and ketones without affecting the carbonyl group are well known, e.g. from P. Gallezot et al. in Catal. Rev.-Sci. Eng. 40 (1&2), (1998) pp. 81-126 and the literature cited therein; P. Claus, Topics in Catalysis 5, (1998), pp. 51-62 and the literature cited therein.

Moreover, the compounds of the formulae (Ia) and (Ib), respectively, where R^{3b} is hydrogen and wherein X is C=O, can be converted to compounds of the formulae (Ic), respectively, wherein X is CH—OH, by hydrogenation of both the C═C double bond and the carbonyl group. The hydrogenation can be performed by analogy to the methods described in EP 1318131 or WO 2006/056435.

The compounds of the formula (Ic), where R^{3a} together with R^{3b} forms CH₂, can be prepared from compounds of the formula (Ia) by subjecting the compound of the formula (Ia) to a cyclopropanation of the C═C double bond.

The cyclopropanation can be easily achieved by reacting the compound of formula (Ia) with diodomethane in the presence of reducing metal agent such as active zinc, e.g. by analogy to a “Simmons-Smith cyclopropanation (see H. E. Simmons, R. D. Smith, J. Am. Chem. Soc. 1958, 80, 5323-5324, J. Am. Chem. Soc., 1959, 81, 4256-4624, for review see also H. E. Simmons et al., Org. React. 1973, 20, 1-131; Charette, Organozinc Reagents 1999, 263-285; Denmark et al. Cycloaddition Reactions in Organic Synthesis 2002, 85-150, Lebel et al. Chem. Rev. 2003, 103, 997-1050). Active zinc may e.g. Zn—Cu or Zn—Ag but also cialkylzinc compounds, such as diethyl zinc (so called Furukawa modification: Furukawa et al, Tetrahedron Lett. 1966, 3353-3354). Instead of active zinc other reducing metals such as samarium or samarium-mercury amalgam may be used instead (Molander modification: Molander et al., J. Org. Chem. 1987, 52, 3942-3944.

### EXAMPLES

### (I) Abbreviations Used

### Fe(acac)₃: Tris(acetylacetonato)iron(III)

hr/hrs: hour(s)
min/mins minutes
RT: room temperature (about 22-23° C.)
MeOH: methanol
NaOMe: sodium methoxide
EtOAc: ethyl acetate
s singlet
d doublet
t triplet
q quartet
m multiplet

### (II) Production Examples

### 2-(1,2,2-trimethylcyclopentyl)acetaldehyde

To a mixture of citral (60:40 mixture of geranial and neral, 50.0 g, 333 mmol) and Fe(acac)₃ (35.0 g, 90 mmol) in 5000 ml MeOH and 1160 ml ethylene glycol 1160 ml was added phenyl silane (65.0 ml, 580 mmol). The resulting mixture was heated to 60° C. with stirring for 1 hr and cooled to room temperature, then diluted with water and brine. The aqueous layer was extracted three times with methyl tert butyl ether. The combined organic layers were dried over sodium sulfate and all volatiles were removed under reduced pressure. The resulting red residue was then dissolved in mixture of ethyl acetate and heptane (10:90 v/v) and filtered through a pad of silica. Removal of the solvent under reduced pressure yielded a light red free flowing product. This was then further purified through column chromatography to give the title product (12.0 g 77 mmole 25%) as a pale yellow liquid.

¹H NMR CDCl₃: δ 9.8 (t 1H), 2.27 (d 2H), 1.88-1.82 (m 1H), 1.7-1.64 (m 3H), 1.63-1.5 (m 1H), 0.98 (s 3H), 0.88 (s 3H), 0.87 (s 3H).

### (III) Preparation of the Compounds of Formulae (Ia), (Ib) and (Ic)

### Example 1: (E)-2-methyl-4-(1,2,2-trimethylcyclopentyl)but-2-enal

To a mixture of 2-(1,2,2-trimethylcyclopentyl)acetaldehyde (10.0 g 64 mmole) and propanal (10.0 g 174 mmol) in 30 ml of isopropyl alcohol was added a 25% solution of NaOMe in MeOH (10 ml, 47 mmol). The resulting solution was stirred for 10 hrs at 60° C. The reaction mass was cooled to RT and 30 ml of water were added. The reaction mass was extracted twice with 75 ml of ethyl acetatel. The combined organic layers were washed with 50 ml brine solution and dried over sodium sulfate. Removing the solvent under reduced pressure yielded crude title compound (8.0 g), which was used without further purification.

¹H NMR CDCl₃: δ 9.35 (s 1H), 6.5 (t 1H), 2.2 (d 2H), 1.7 (s 3H), 1.7-1.4 (6H), 0.9-0.8 (s 9H)

### Example 2: (E)-2-ethyl-4-(1,2,2-trimethylcyclopentyl)but-2-enal

To a mixture of 2-(1,2,2-trimethylcyclopentyl)acetaldehyde (6.5 g 33 mmol) and butanal (8.0 g 111 mmol) in 25 ml of isopropyl alcohol was added a 25% solution of NaOMe in MeOH (7.5 ml, 38 mmol). The resulting solution was stirred for 10 hrs at 80° C. The reaction mass was cooled to RT and 30 ml of water were added. The reaction mass was extracted twice with 50 ml of ethyl acetate. The combined organic layers were washed with 50 ml brine solution and dried over sodium sulfate. Removing the solvent under reduced pressure yielded crude title compound (8.0 g), which was used without further purification.

¹H NMR CDCl₃: δ 9.35 (s 1H), 6.5 (t 1H), 2.2 (d 2H), 1.7 (q 2H), 1.7-1.4 (6H), 0.9-0.8 (s 9H), 0.8-0.75 (s 3H).

### Example 3: (E)-2-ethyl-4-(1,2,2-trimethylcyclopentyl)but-2-en-1-ol

A mixture of crude (E)-2-ethyl-4-(1,2,2-trimethylcyclopentyl)but-2-enal of example 2 (8.0 g, 38 mmol) and methanol (25 ml) was cooled to 0° C. and NaBH₄ (1.5 g, 39 mmol) was added slowly. The reaction mass was stirred at 20° C. for 30 min. 50 ml of water were added and the reaction mass was extracted twice with ethyl acetate (75 ml). The combined organic layers were dried on sodium sulfate. Removing the solvent under reduced pressure gave crude (E)-2-ethyl-4-(1,2,2-trimethylcyclopentyl)but-2-en-1-ol (6.5 g). The crude product was then purified by column chromatography to give pure E)-2-ethyl-4-(1,2,2-trimethylcyclopentyl)but-2-en-1-ol (1.2 g)

¹H NMR CDCl₃: δ 5.4-5.45 (t 1H), 3.95 (s 2H), 2.2-2.1 (q 2H), 2.0-2.2 (d 2H), 1.7-1.4 (6H), 1.0-0.95 (t 3H), 0.9-0.8 (s 6H), 0.8-0.75 (s 3H).

### Example 4: (E)-5-(1,2,2-trimethylcyclopentyl)pent-3-en-2-one

To a mixture of 2-(1,2,2-trimethylcyclopentyl)acetaldehyde (10.0 g 64.94 mmol), acetone (50.0 ml) and molecular sieves A4 lithium hydroxide monohydrate (4.2 g 100.0 mmol) was added. The reaction mixture was stirred at 60° C. for 6 h and then it was cooled to RT and the resulting solid was filtered off. The filtrate was diluted with 100.0 ml ethyl acetate and washed with 50.0 ml 1N HCl and 50.0 ml water. The resulting organic layer was dried over sodium sulfate. Removing the solvent under reduced pressure gave crude (E)-5-(1,2,2-trimethylcyclopentyl)pent-3-en-2-one (10.0 g). The crude product was then purified by column chromatography (silicagel; ethyl acetate:heptane 1:9 v/v) to give pure (E)-5-(1,2,2-trimethylcyclopentyl)pent-3-en-2-one (6.0 g) (Yield 55%).

¹H NMR CDCl₃: δ 6.8-6.35 (q 1H), 6.0-5.95 (d1H), 2.2-2.1 (d 2H), 1.7 (s 3H), 2.1-2.0 (d 2H), 1.6-1.4 (6H), 0.9-0.7 (9H)

### Example 5: (E)-4-methyl-6-(1,2,2-trimethylcyclopentyl)hex-4-en-3-one

To a mixture of 2-(1,2,2-trimethylcyclopentyl)acetaldehyde (10.0 g 64.94 mmol), diethylketone (50.0 ml) and molecular sieves A4 lithium hydroxide monohydrate (4.2 g 100.0 mmol) was added. The reaction mixture was stirred at 100° C. for 8 hrs and then it was cooled to RT and the resulting solid was filtered off. The filtrate was diluted with 100.0 ml ethyl acetate and washed with 50.0 ml 1N HCl and 50.0 ml water. The resulting organic layer was dried over sodium sulfate. Removing the solvent under reduced pressure gave crude (E)-4-methyl-6-(1,2,2-trimethylcyclopentyl)hex-4-en-3-one (12.0 g). The crude product was then purified by column chromatography (silicagel; ethyl acetate:heptane 1:9 v/v) to give pure (E)-4-methyl-6-(1,2,2-trimethylcyclopentyl)hex-4-en-3-one (6.0 g) (Yield 52%).

¹H NMR CDCl₃: δ 6.8 (d 1H), 3.8 (d1H), 2.7 (t 2H), 2.1 (d 2H), 1.7 (s 3H), 1.6-1.4 (6H), 1.0 (t 3H) 0.9-0.8 (9H)

### Example 6: (E)-4-methyl-6-(1,2,2-trimethylcyclopentyl)hex-4-en-3-ol

(E)-4-methyl-6-(1,2,2-trimethylcyclopentyl)hex-4-en-3-one (5.0 g 22.52 mmol) was dissolved in methanol (25.0 ml) and NaBH₄ (1.0 g 26.31 mmol) was added slowly at 0° C. within 15 min. The reaction mixture was stirred for 1 h at 10° C. Then 75.0 ml water was added and the obtained mixture was extracted twice with 50.0 ml ethyl acetate. Removing the solvent under reduced pressure gave crude (E)-4-methyl-6-(1,2,2-trimethylcyclopentyl)hex-4-en-3-ol (4.5 g). The crude product was then purified by column chromatography (silicagel; ethyl acetate:heptane 1.5:8.5 v/v) to give pure (E)-4-methyl-6-(1,2,2-trimethylcyclopentyl)hex-4-en-3-ol (3.0 g) (Yield 75%).

¹H NMR CDCl₃: δ 5.4 (t 1H), 3.8 (d1H), 1.9 (d 2H), 1.7 (s 3H), 1.6-1.4 (6H), 1.4 (q 2H) 0.9-0.8 (s 9H), 0.7 (q 3H).

### Example 7: (E)-2-methyl-4-(1,2,2-trimethylcyclopentyl) but-2-en-1-ol

10.0 g (0.0515 mol) of (E)-2-methyl-4-(1,2,2-trimethylcyclopentyl)but-2-enal was dissolved in methanol (50.0 ml) and the solution was cooled to 0° C. To this solution 2.0 g (0.06 mol) of sodium borohydride was added in portions while maintaining temperature at 0-5° C. The reaction mixture was stirred at 0° C. for 20 mins. After completion of reaction, the reaction mixture was concentrated in vacuo to remove methanol. The residue was taken in 150.0 ml ethyl acetate and washed with water (2×100 ml). The organic layer was washed with brine, dried over sodium sulphate and concentrated in vacuo to yield 7.8 g of crude (E)-2-methyl-4-(1,2,2-trimethylcyclopentyl) but-2-en-1-ol. The crude product was purified by flash column chromatography (Eluent: 25:75 Ethyl acetate/heptanes) to get 3.1 g of pure (E)-2-methyl-4-(1,2,2-trimethylcyclopentyl) but-2-en-1-ol.

¹H NMR, 300 MHz, CDCl₃, δ ppm 5.51-5.46 (t, 1H), 4.02 (s, 2H), 1.97-1.94 (d, 2H), 1.66 (s, 3H), 1.62-1.40 (m, 6H), 0.88 (s, 3H), 0.87 (s, 3H), 0.80 (s, 3H).

### Example 8: [1-methyl-2-[(1,2,2-trimethylcyclopentyl)methyl]cyclopropyl]methanol

Under nitrogen atmosphere, diethyl zinc (1.0 M in hexanes) (7.56 g, 61.2 mmol) was added to 1,2-dichloroethane (240.0 ml) at RT. To this mixture diiodomethane (31.95 g, 119.3 mmol) was added dropwise over a period of 2 h. The reaction mixture was stirred for 30 mins. at RT. (E)-2-methyl-4-(1,2,2-trimethylcyclopentyl) but-2-en-1-ol (3.0 g, 15.3 mmol) was added dropwise to the thus obtained mixture and the resulting mixture was stirred for 4 h at RT. The resulting reaction mixture was poured into an aqueous solution of potassium carbonate (200.0 ml, 20% b.w.) and the mixture was filtered through a pad of Celite in a sintered funnel. The organic layer was separated and dried over sodium sulfate. The solvent was stripped off under reduced pressure to yield crude title compound (3.0 g). The crude product was then purified by column chromatography (SiO₂, Eluent: 5% EtOAc/Heptanes) to give pure [1-methyl-2-[(1,2,2-trimethylcyclopentyl) methyl] cyclopropyl] methanol (2.0 g).

¹H NMR DMSO: δ 4.59 (br 1H), 3.25 (s 2H), 1.89-1.50 (m 6H), 1.29 (d 2H), 1.19 (s 3H), 1.04 (s 3H), 1.01 (s 3H), 0.94 (s 3H), 0.77-0.63 (m 2H), 0.00 (m 1H).

### Example 9: [1-ethyl-2-[(1,2,2-trimethylcyclopentyl)methyl]cyclopropyl]methanol

Under nitrogen atmosphere, diethyl zinc (1.0 M in hexanes) (7.07 g, 57.1 mmol) was added to 1,2-dichloroethane (240.0 ml) at RT. To this mixture diiodomethane (29.8 g, 111.4 mmol) was added dropwise over a period of 2 h. The reaction mixture was stirred for 30 mins. at RT. (E)-2-ethyl-4-(1,2,2-trimethylcyclopentyl) but-2-en-1-ol (3.0 g, 14.2 mmol) was added dropwise to the thus obtained mixture and the resulting mixture was stirred for 4 h at RT. The resulting reaction mixture was poured into an aqueous solution of potassium carbonate (200.0 ml, 20% b.w.) and the mixture was filtered through a pad of Celite in a sintered funnel. The organic layer was separated and dried over sodium sulfate. The solvent was stripped off under reduced pressure to yield crude title compound (3.0 g). The crude product was then purified by column chromatography (SiO₂, Eluent: 5% EtOAc/Heptanes) to give pure [1-ethyl-2-[(1,2,2-trimethylcyclopentyl) methyl] cyclopropyl] methanol (1.5 g).

¹H NMR DMSO: δ 4.20 (br 1H), 3.14-3.10 (d 1H), 2.89-2.85 (m 1H), 1.38-1.21 (m 8H), 1.04 (m 1H), 0.88 (m 1H), 0.77-0.61 (12H), 0.46-0.41 (m 1H), 0.30-0.22 (m 1H), −0.32-0.37 (m 1H).

### (IV) Scent Strip Tests

To evaluate the quality and intensity of the odor of the compounds, scent strip tests were performed.

For this purpose strips of absorbent paper were dipped into solution containing 10 wt. % of the respective compound in ethanol. After evaporation of the solvent (about 30 sec.) the scent impression was olfactorically evaluated.

Scent Strip Test Result:

Example 1: (E)-2-methyl-4-(1,2,2-trimethylcyclopentyl)but-2-enal Odor impression: woody, ambra, floral aldehydic.

Example 2: (E)-2-ethyl-4-(1,2,2-trimethylcyclopentyl)but-2-enal Odor impression: sweet, fruity.

Example 3: (E)-2-ethyl-4-(1,2,2-trimethylcyclopentyl)but-2-en-1-ol Odor impression: typical clean sandalwood, creamy, urinous

Volatility:

long lasting on blotter (>48 h)

Example 4: (E)-5-(1,2,2-trimethylcyclopentyl)pent-3-en-2-one Odor impression: woody, ambra, sweet, milky.

Example 5: (E)-4-methyl-6-(1,2,2-trimethylcyclopentyl)hex-4-en-3-one Odor impression: woody, ambra, androstenone.

Example 6: (E)-4-methyl-6-(1,2,2-trimethylcyclopentyl)hex-4-en-3-ol Odor impression: Sandalwood, urinous, milky.

Example 8: [1-methyl-2-[(1,2,2-trimethylcyclopentyl)methyl]cyclopropyl]methanol

Odor impression (scala from 1-6)

Intensity 5

sandalwood 6

cream 3

sweet floral 4

fresh 3

water 3

aldehydic substantivity 6

Example 9: [1-ethyl-2-[(1,2,2-trimethylcyclopentyl)methyl]cyclopropyl]methanol

Odor impression (scala from 1-6)

Intensity 4

sandalwood 6

cream 4

woody 3

aldehydic substantivity 6

1.-14. (canceled)

15. A compound of the general formula (Ia) or (Ic) 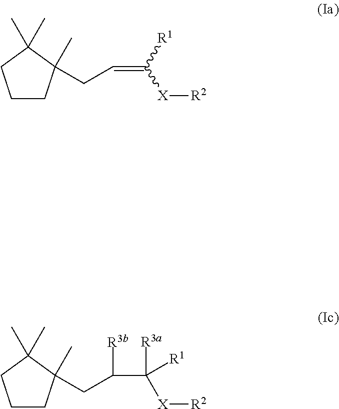
or mixtures thereof, in which
X is CH—OH or C═O;
R¹ is selected from the group consisting of hydrogen, methyl and ethyl,
R² is selected from the group consisting of hydrogen, methyl and ethyl,
R^{3b} together with R^{3a} forms CH₂;
including the stereoisomers thereof.

16. The compound or mixture of claim 15, wherein X is CO.

17. The compound or mixture of claim 15, wherein X is CH—OH.

18. The compound or mixture of claim 15, which is a compound of formula (Ia), a mixture of compounds of formula (Ia) or a mixture of at least one compound of formula (Ia) with one or more compounds of the formulae (Ic).

19. The compound or mixture of claim 15, which is a compound of formula (Ic), a mixture of compounds of formula (Ic) or a mixture of at least one compound of formula (Ic) with one or more compounds of the formulae (Ia).

20. The compound of claim 15, which is selected from the group consisting of
4-(1,2,2-trimethylcyclopentyl)but-2-enal,
2-methyl-4-(1,2,2-trimethylcyclopentyl)but-2-enal,
2-ethyl-4-(1,2,2-trimethylcyclopentyl)but-2-enal,
5-(1,2,2-trimethylcyclopentyl)pent-3-en-2-one,
3-methyl-5-(1,2,2-trimethylcyclopentyl)pent-3-en-2-one,
3-ethyl-5-(1,2,2-trimethylcyclopentyl)pent-3-en-2-one,
6-(1,2,2-trimethylcyclopentyl)hex-4-en-3-one,
4-methyl-6-(1,2,2-trimethylcyclopentyl)hex-4-en-3-one,
4-(1,2,2-trimethylcyclopentyl)but-2-en-1-ol,
2-methyl-4-(1,2,2-trimethylcyclopentyl)but-2-en-1-ol,
2-ethyl-4-(1,2,2-trimethylcyclopentyl)but-2-en-1-ol,
5-(1,2,2-trimethylcyclopentyl)pent-3-en-2-ol,
3-methyl-5-(1,2,2-trimethylcyclopentyl)pent-3-en-2-ol,
3-ethyl-5-(1,2,2-trimethylcyclopentyl)pent-3-en-2-ol,
6-(1,2,2-trimethylcyclopentyl)hex-4-en-3-ol, and
4-methyl-6-(1,2,2-trimethylcyclopentyl)hex-4-en-3-ol,
and their stereoisomers and mixtures thereof.

21. The compound of claim 15, which is selected from the group consisting of
[E-1-methyl-2-[(1,2,2-trimethylcyclopentyl)methyl]cyclopropylmethanol;
[E-1-ethyl-2-[(1,2,2-trimethylcyclopentyl)methyl]cyclopropylmethanol;
[Z-1-methyl-2-[(1,2,2-trimethylcyclopentyl)methyl]cyclopropylmethanol; and
[Z-1-ethyl-2-[(1,2,2-trimethylcyclopentyl)methyl]cyclopropylmethanol.

22. A fragrance or a flavor which comprises the compound or mixture of claim 15.

23. The fragrance or a flavor of claim 22, where the compounds of formulae (Ia) or (Ic) or a mixture thereof is incorporated into a composition, further comprising a carrier.

24. A method of imparting or modifying a scent or a flavor to a composition, which method comprises incorporating the compound of the formulae (Ia) or (Ic) as defined in claim 15 or a mixture thereof into a composition in such an amount that imparts or modifies the scent or flavor of the composition.

25. A fragrance containing composition and/or a fragrance material, which contains at least one compound selected from compounds of the general formulae (Ia) or (Ic) as defined in claim 15 or a mixture thereof and a carrier material.

26. A process for preparing the compound of the formula (Ia) as defined in claim 15, which comprises
i. providing 2-(1,2,2-trimethylcyclopentyl)acetaldehyde of the formula (II) 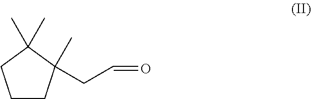
ii. reacting 2-(1,2,2-trimethylcyclopentyl)acetaldehyde with a compound of the formula (IIIa) 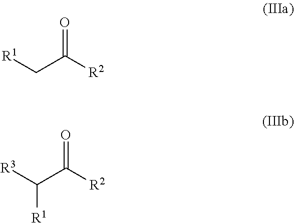
wherein R¹ and R² are as defined herein, under conditions of an aldol condensation to obtain a compound of the formula (Ia), wherein X is C═O, and optionally
iii. subjecting the compound of the formula (Ia), wherein X is C═O to a reduction reaction of the carbonyl group to a hydroxyl group to obtain a compound of the formulae (Ia), wherein X is CH—OH.

27. The process of claim 26, wherein 2-(1,2,2-trimethylcyclopentyl)acetaldehyde of formula (II) is provided by reaction of citral with a reducing agent in the presence of an iron-containing catalyst.

28. A process for preparing a compound of the formula (Ic) as defined in claim 15, which comprises providing a compound of the formula (Ia) by the process of claim 26 or 27 and subjecting the compound of the formula (Ia) to a cyclopropanation of the C═C double bond.
